(19) United States
(12) Patent Application Publication
(10) Pub. No.: US 2005/0043753 A1
(43) Pub. Date: Feb. 24, 2005

(54) Apparatus and method for an ultrasonic medical device to treat peripheral artery disease

(75) Inventors: Robert A. Rabiner, North Reading, MA (US); Bradley A. Hare, Chelmsford, MA (US); Craig T. Kelley, Lexington, MA (US)

Correspondence Address: PALMER & DODGE, LLP;RICHARD B. SMITH, 111 HUNTINGTON AVENUE, BOSTON, MA, 02199, US

(73) Assignee: OmniSonics Medical Technologies, Inc.

(21) Appl. No.: 10/960,865

(22) Filed: Oct. 7, 2004

Related U.S. Application Data
(63) Continuation-in-part of application No. 10/665,445, filed on Sep. 19, 2003.
Application No. 10/665,445 is a continuation of application No. 09/776,015, filed on Feb. 2, 2001, now Pat. No. 6,652,547.
Application No. 09/776,015 is a continuation-in-part of application No. 09/618,352, filed on Jul. 19, 2000, now Pat. No. 6,551,337.
(60) Provisional application No. 60/178,901, filed on Jan. 28, 2000.
(60) Provisional application No. 60/157,824, filed on Oct. 5, 1999.

(51) Int. Cl. A61B 17/22

(52) U.S. Cl. 606/159; 606/169

(57) ABSTRACT

An apparatus and method for an ultrasonic medical device to treat peripheral artery disease. The ultrasonic medical device comprises an ultrasonic probe having a proximal end, a distal end and a longitudinal axis therebetween. The ultrasonic probe is inserted into an insertion point in a leg opposite the leg having an occlusional deposit and is moved adjacent to the occlusional deposit. An ultrasonic energy source is activated to generate a transverse ultrasonic vibration along at least a portion of the longitudinal axis of the ultrasonic probe. The transverse ultrasonic vibration creates a plurality of transverse nodes and a plurality of transverse anti-nodes along the longitudinal axis of the ultrasonic probe, generating cavitation in a medium surrounding the ultrasonic probe to ablate the occlusional deposit causing peripheral artery disease.

### RELATED APPLICATIONS

This application is a continuation-in-part of application Ser. No. 10/665,445, filed Sep. 19, 2003, which is a continuation of application Ser. No. 09/776,015, filed Feb. 2, 2001, now U.S. Pat. No. 6,652,547, which is a continuation-in-part of application Ser. No. 09/618,352, filed Jul. 19, 2000, now U.S. Pat. No. 6,551,337, which claims benefit of Provisional Application Ser. No. 60/178,901, filed Jan. 28, 2000, and claims benefit of Provisional Application Ser. No. 60/157,824, filed Oct. 5, 1999, the entirety of all these applications are hereby incorporated herein by reference.

### FIELD OF THE INVENTION

The present invention relates to medical devices, and more importantly to an apparatus and a method for an ultrasonic medical device to treat peripheral artery disease.

### BACKGROUND OF THE INVENTION

Peripheral artery disease (PAD) affects approximately twelve million adults in the United States. The incidence of peripheral artery disease is increased for people over the age of seventy, with approximately twenty percent of Americans over seventy affected with peripheral artery disease. By contrast, less than eight percent of Americans under age seventy are affected by peripheral artery disease. Peripheral artery disease increases the risk of heart attack, stroke, amputation of lower extremity limbs and, in some cases, death.

Peripheral artery disease is a form of atherosclerosis in which occlusional deposits build up along the artery walls and reduce blood circulation. Tissues throughout the body, which receive blood from the arteries, need oxygen from the blood that is delivered through the arteries. The buildup of the occlusional deposits along the artery walls reduces the cross sectional area through which the blood flows, therefore depriving the tissues of oxygen and increasing the potential of damage to the tissues. The arteries lose elasticity and are unable to dilate to allow for greater blood flow when needed.

Peripheral artery disease occurs in the hundreds of arteries outside of the heart, better known as the peripheral arteries. The most common location for peripheral artery disease is in the peripheral arteries in the legs and feet. Within the legs, the most common location of peripheral artery disease is in the iliac, femoral and popliteal arteries. Peripheral artery disease also occurs in the arms and there has been a low frequency of peripheral artery disease in the heart and the brain. In the early stages of peripheral artery disease in the leg, cramping or fatigue in the legs, calves and buttocks occurs during activity, leading to a type of pain known as claudication. Despite oxygen reaching the muscles during a rest period, a pain cycle known as intermittent claudication often provides the first warning sign of peripheral artery disease. Advanced cases of peripheral artery disease may lead to gangrene, ulcers and leg amputation.

Other areas in the body where atherosclerosis produces symptoms of peripheral artery disease are the cerebrovascular arteries, renal arteries and mesenteric arteries. Blockage in the cerebrovascular arteries, also known as the brain arteries, leads to cerebrovascular disease. Cerebrovascular disease is a leading cause of stroke and disability. Stenosis in the renal arteries, also known as the kidney arteries, is a major cause of high blood pressure and renal failure requiring dialysis or kidney transplant. Peripheral artery disease of the mesenteric arteries, also known as the intestinal arteries, leads to mesenteric arterial disease. Mesenteric arterial disease is less common but can cause severe pain, weight loss and even death from intestinal gangrene.

Prior art methods of treating peripheral artery disease are invasive, increase the probability of future problems and offer temporary relief mechanisms that do not effectively treat the cause of the peripheral artery disease. Pharmacological agents can be used to enlarge or dilate the affected artery, but pharmacological agents often produce harmful health side effects. A balloon angioplasty can be performed in an attempt to open an artery, but a balloon angioplasty places high stresses on the vasculature that may compromise the integrity of the vasculature. Surgical procedures including removal of the lining of the artery (endarterectomy) or repair or replacement of the vessel (grafting) are invasive procedures that subject the patient to risks and trauma.

U.S. Pat. No. 6,522,929 to Swing discloses a treatment of peripheral vascular disease using an electrical stimulator and acupuncture needles. A plurality of the Swing acupuncture needles are placed at specific acupuncture points and a current is passed through the acupuncture needles. The application of the current to the blood vessels with the Swing device increases the blood flow to the vessels, allowing more oxygen and body nutrients to treat the peripheral vascular disease. The Swing device causes a stinging pain and trauma to the patient. In addition, the Swing device does not directly treat the occlusive deposits causing the peripheral vascular disease.

U.S. Pat. No. 5,231,080 to Scholkens discloses a method for treatment of peripheral vessel disease by administration of angiotensin converting enzyme inhibitors. Scholkens discloses administration of angiotensin converting enzyme inhibitors to prevent platelet aggregation. The Scholkens inhibitor does not remove the occlusional deposits comprising the peripheral vessel disease, but rather comprises administration of an inhibitor to prevent platelet aggregation.

The prior art does not provide a solution for effectively preventing and treating peripheral artery disease. The prior art does not remove the occlusions or deposits comprising the peripheral artery disease and the prior art presents adverse consequences to the patient. Therefore, there remains a need in the art for an apparatus and a method of preventing and treating peripheral artery disease that effectively removes the occlusional deposits in a safe, effective and time efficient manner.

### SUMMARY OF THE INVENTION

The present invention is an ultrasonic medical device for resolving peripheral artery disease comprising: an ultrasonic probe having a proximal end, a distal end and a longitudinal axis between the proximal end and the distal end; a transducer creating a transverse ultrasonic vibration along at least a portion of the longitudinal axis of the ultrasonic probe; a coupling engaging the proximal end of the ultrasonic probe to a distal end of the transducer; and an ultrasonic energy source engaged to the transducer that produces an ultrasonic energy. The transverse ultrasonic vibration produces a plurality of transverse nodes and a plurality of transverse anti-nodes along at least a portion of the longitudinal axis of the ultrasonic probe to ablate an occlusional deposit causing peripheral artery disease.

The present invention is an ultrasonic medical device for treating peripheral artery disease. The ultrasonic medical device includes an ultrasonic probe having a proximal end, a distal end terminating in a probe tip and a longitudinal axis between the proximal end and the distal end. The ultrasonic medical device also includes a transducer that converts electrical energy into mechanical energy, creating a transverse ultrasonic vibration along the longitudinal axis of the ultrasonic probe, and a coupling engaging the proximal end of the ultrasonic probe and a distal end of the transducer. The transverse ultrasonic vibration generates a plurality of transverse nodes and a plurality of transverse anti-nodes along at least a portion of the longitudinal axis of the ultrasonic probe, creating cavitation in a medium surrounding the ultrasonic probe to ablate an occlusional deposit and treat peripheral artery disease.

The present invention is a method of treating peripheral artery disease. An ultrasonic medical device comprising an ultrasonic probe having a proximal end, a distal end terminating in a probe tip and a longitudinal axis between the proximal end and the distal end is provided. The ultrasonic probe is inserted into a vasculature and moved adjacent to an occlusional deposit in a peripheral artery. The ultrasonic probe is placed in communication with the occlusional deposit and an ultrasonic energy source engaged to the ultrasonic probe is activated to generate a transverse ultrasonic vibration along at least a portion of the longitudinal axis of the ultrasonic probe. The transverse ultrasonic vibration creates a plurality of transverse anti-nodes along a portion of the longitudinal axis of the ultrasonic probe.

The present invention is a method of ablating an occlusional deposit to treat peripheral artery disease comprising: providing an ultrasonic medical device comprising an ultrasonic probe having a proximal end, a distal end and a longitudinal axis between the proximal end and the distal end; inserting the ultrasonic probe into a femoral artery; moving the ultrasonic probe into a peripheral artery; placing the ultrasonic probe in communication with an occlusional deposit in the peripheral artery; and activating an ultrasonic energy source engaged to the ultrasonic probe to produce an electric signal that drives a transducer of the ultrasonic medical device to produce a transverse ultrasonic vibration of the ultrasonic probe, wherein the transverse ultrasonic vibration produces cavitation in a medium surrounding the ultrasonic probe to ablate the occlusional deposit.

The present invention provides an apparatus and a method for an ultrasonic medical device to treat peripheral artery disease. An ultrasonic probe is placed in communication with an occlusional deposit causing peripheral artery disease and a transverse ultrasonic vibration along at least a portion of the longitudinal axis of the ultrasonic probe ablates the occlusional deposit. The present invention provides an ultrasonic medical device for treating peripheral artery disease that is simple, user-friendly, time efficient, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a side plan view of an ultrasonic probe of the present invention inserted contralaterally into an external iliac artery of a lower limb of a patient.

FIG. 2 is a side plan view of an ultrasonic probe of the present invention inserted contralaterally into a superficial femoral artery of a lower limb of a patient.

FIG. 3 is a side plan view of an ultrasonic probe of the present invention inserted ipsilaterally into an artery of a lower limb of a patient.

FIG. 4 is a side plan view of an ultrasonic medical device of the present invention capable of ablating an occlusional deposit to treat peripheral artery disease.

FIG. 5 is a side plan view of an ultrasonic probe of the present invention having an approximately uniform diameter from a proximal end of the ultrasonic probe to the distal end of the ultrasonic probe.

FIG. 6 shows a side plan view of an ultrasonic probe of the present invention showing a plurality of transverse nodes and a plurality of transverse anti-nodes along a portion of a longitudinal axis of the ultrasonic probe.

FIG. 7 is a view of a leg of a patient showing a plurality of peripheral arteries with an occlusional deposit in an external iliac artery of a leg.

FIG. 8 is an enlarged view of a portion of an ultrasonic probe of the present invention inserted into an external iliac artery and being moved toward an occlusional deposit in the external iliac artery of a leg.

FIG. 9 is an enlarged view of an ultrasonic probe of the present invention in communication with an occlusional deposit in an external iliac artery of a leg.

FIG. 10 is an enlarged view of an ultrasonic probe of the present invention showing a plurality of transverse anti-nodes in communication with an occlusional deposit in an external iliac artery of a leg.

FIG. 11 is an enlarged view of an ultrasonic probe in communication with a partially ablated occlusional deposit in an external iliac artery.

FIG. 12 is an enlarged view of an external iliac artery after ablation of an occlusional deposit using an ultrasonic probe of the present invention.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using an ultrasonic medical device to treat peripheral artery disease. The ultrasonic medical device comprises an ultrasonic probe having a proximal end, a distal end and a longitudinal axis therebetween, a transducer creating a transverse ultrasonic vibration along at least a portion of the longitudinal axis of the ultrasonic probe, a coupling engaging the proximal end of the ultrasonic probe to a distal end of the transducer and an ultrasonic energy source engaged to the transducer that produces an ultrasonic energy. The transverse ultrasonic vibration generates a plurality of transverse nodes and a plurality of transverse anti-nodes along at least a portion of the longitudinal axis of the ultrasonic probe, creating cavitation in a medium surrounding the ultrasonic probe to ablate an occlusional deposit and treat peripheral artery disease.

While the present invention is an apparatus and a method for using an ultrasonic medical device to treat peripheral artery disease, the ultrasonic medical device of the present invention can also be used to treat peripheral vascular disease, including arterial or venous disease. Atheroma, fatty deposits in the intima of the vasculature, do not generally occur on the venous side where the usual problem is thrombosis arising from an inability to get the blood back to the lungs (i.e., venous valve damage, external trauma, stenosis). On the arterial side, the occlusional deposit or thrombus results from damage to the vasculature by the atheroma or by narrowing of the vessel as the atheroma builds up. The ultrasonic medical device of the present invention may be used to treat peripheral artery disease, peripheral vascular disease and other arterial or venous diseases.

The following terms and definitions are used herein:

“Ablate” as used herein refers to removing, clearing, destroying or taking away an occlusional deposit. “Ablation” as used herein refers to a removal, clearance, destruction, or taking away of the occlusional deposit.

“Anti-node” as used herein refers to a region of a maximum energy emitted by an ultrasonic probe at or adjacent to a specific location along a longitudinal axis of the ultrasonic probe.

“Node” as used herein refers to a region of a minimum energy emitted by an ultrasonic probe at or adjacent to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe).

“Occlusional deposit” as used herein refers to a collection of a matter including, but not limited to, a group of similar cells, intravascular blood clots, occlusions, thrombus, plaque, biological material, fibrin, calcified plaque, atheroma, calcium deposits, biological materials, atherosclerotic plaque, fatty deposits, adipose tissues, atherosclerotic cholesterol buildup, fibrous material buildup, arterial stenoses, minerals, high water content tissues, platelets, cellular debris, wastes and other occlusive materials.

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Vasculature” as used herein refers to the entire circulatory system for the blood supply including the venous system, the arterial system and the associated vessels, arteries, veins, capillaries, blood, and the heart. The arterial system is the means by which blood with oxygen and nutrients is transported to tissues. The venous system is the means by which blood with carbon dioxide and metabolic by-products is transported for excretion.

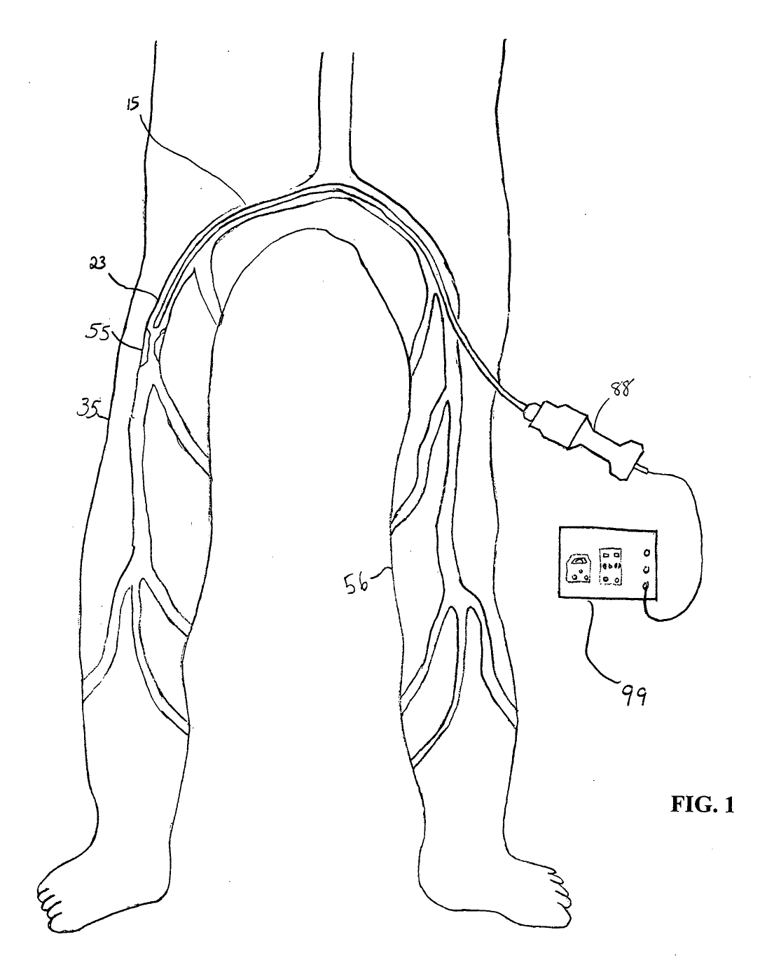

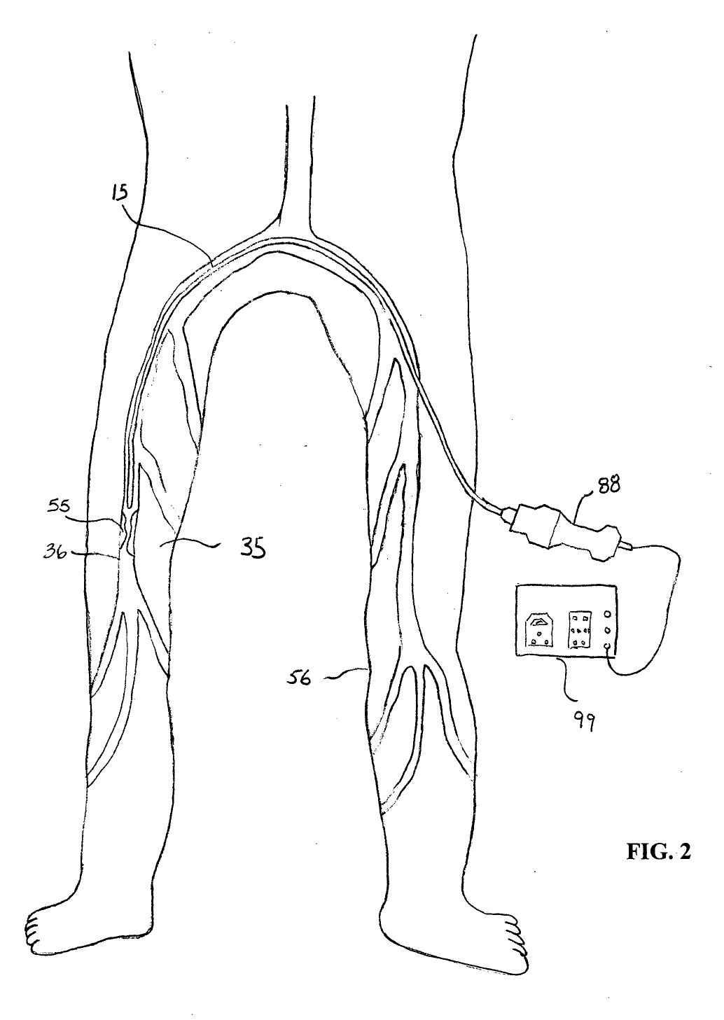

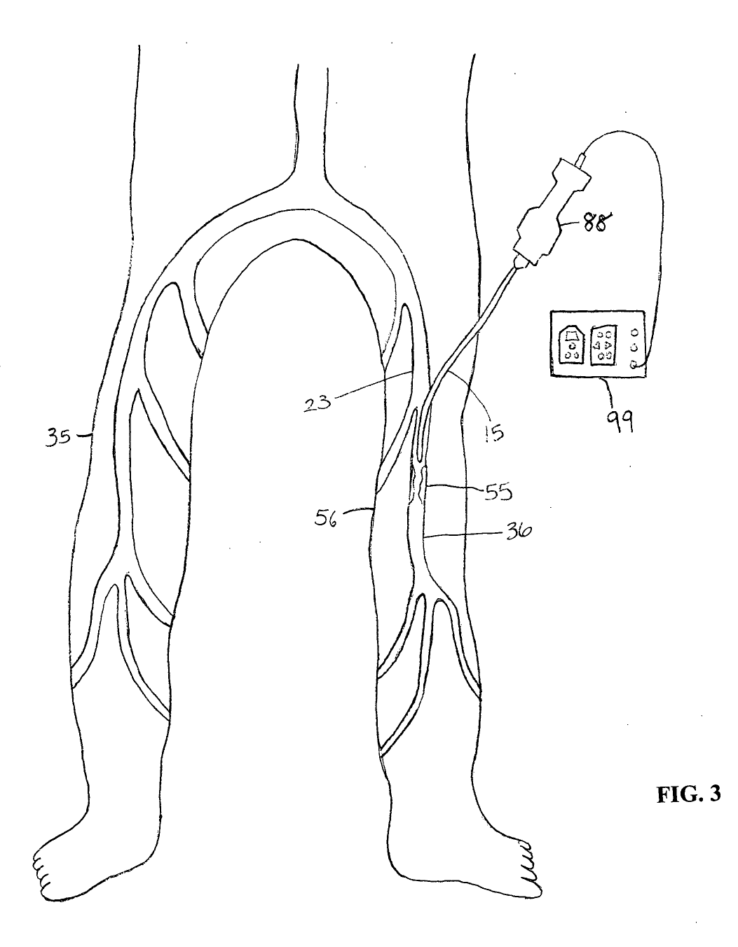

An ultrasonic probe of an ultrasonic medical device of the present invention capable of ablating an occlusional deposit 55 to treat peripheral artery disease is illustrated generally at 15 in FIG. 1. FIG. 1 shows the ultrasonic probe 15 inserted at an upper part of a leg 35 and adjacent to the occlusional deposit 55 in an external iliac artery 23. In an embodiment of the present invention shown in FIG. 1, the ultrasonic probe 15 is inserted into a leg 56 opposite the leg 35 having the occlusional deposit 55 for a contralateral approach. A flexibility of the ultrasonic probe 15 allows the ultrasonic probe 15 to be navigated within the vasculature to the external iliac artery 23. FIG. 2 shows the ultrasonic probe 15 inserted contralaterally in an upper part of the leg 56 and moved into the leg 35 adjacent to the occlusional deposit 55 in the superficial femoral artery 36 of the leg 35. FIG. 3 shows an embodiment of the present invention in which the ultrasonic probe 15 is inserted ipsilaterally at an upper part of the leg 56 to treat the occlusional deposit 55 in the superficial femoral artery 36 of the leg 56. The ipsilateral approach shown in FIG. 3 inserts the ultrasonic probe 15 into the vasculature in a leg on the same side of the body as the occlusional deposit 55 to be treated.

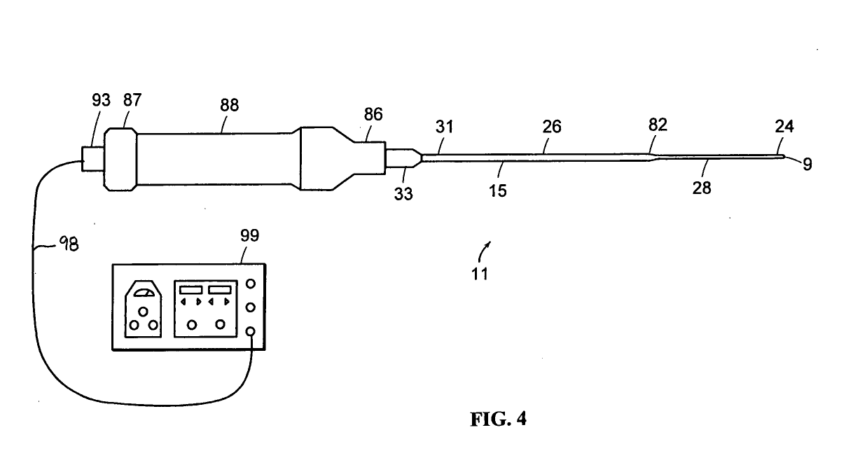

FIG. 4 shows an ultrasonic medical device capable of ablating an occlusional deposit 55 to treat peripheral artery disease and prevent ischemia of tissues and muscles served by the peripheral arteries. In a preferred embodiment of the present invention, the ultrasonic probe 15 is used to ablate an occlusional deposit in the leg 35 of a patient. The ultrasonic medical device 11 includes an ultrasonic probe 15 which is coupled to an ultrasonic energy source or generator 99 for the production of an ultrasonic energy. A handle 88, comprising a proximal end 87 and a distal end 86, surrounds a transducer within the handle 88. The transducer, having a proximal end engaging the ultrasonic energy source 99 and a distal end coupled to a proximal end 31 of the ultrasonic probe 15, transmits the ultrasonic energy to the ultrasonic probe 15. A connector 93 and a connecting wire 98 engage the ultrasonic energy source 99 to the transducer. The ultrasonic probe 15 includes the proximal end 31, a distal end 24 that ends in a probe tip 9 and a longitudinal axis between the proximal end 31 and the distal end 24. In a preferred embodiment of the present invention shown in FIG. 4, a diameter of the ultrasonic probe decreases from a first defined interval 26 to a second defined interval 28 along the longitudinal axis of the ultrasonic probe 15 over a transition 82. A coupling 33 that engages the proximal end 31 of the ultrasonic probe 15 to the transducer within the handle 88 is illustrated generally in FIG. 4. In a preferred embodiment of the present invention, the coupling is a quick attachment-detachment system. An ultrasonic medical device with a rapid attachment and detachment means is described in the Assignee's U.S. Pat. No. 6,695,782 and Assignee's co-pending patent applications U.S. Ser. No. 10/268,487 and U.S. Ser. No. 10/268,843, which further describe the quick attachment-detachment system and the entirety of these patents and patent applications are hereby incorporated herein by reference.

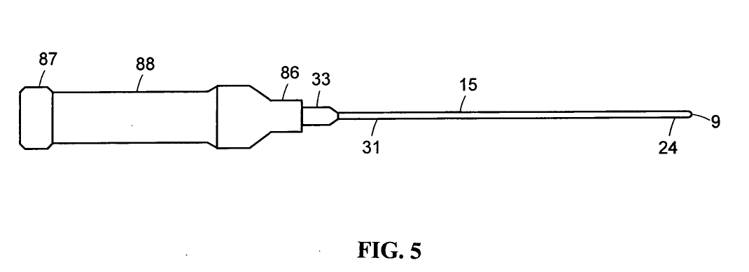

FIG. 5 shows an embodiment of the ultrasonic probe 15 of the present invention where the diameter of the ultrasonic probe 15 is approximately uniform from the proximal end 31 of the ultrasonic probe 15 to the distal end 24 of the ultrasonic probe 15.

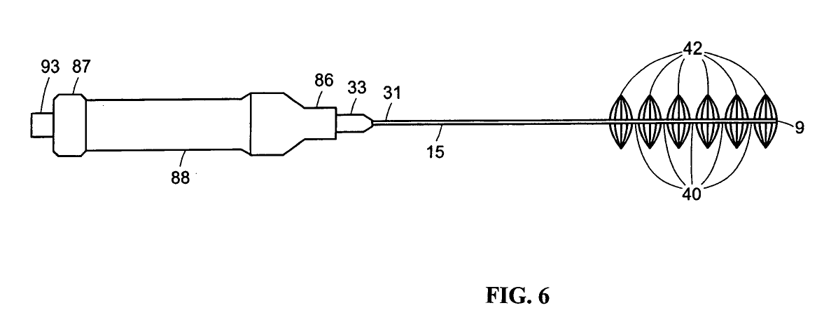

FIG. 6 shows a side plan view of an ultrasonic probe 15 of the present invention showing a plurality of transverse nodes 40 and a plurality of transverse anti-nodes 42 along a portion of a longitudinal axis of the ultrasonic probe 15.

In a preferred embodiment of the present invention, the ultrasonic probe 15 is a wire. In an embodiment of the present invention, the ultrasonic probe 15 is elongated. In an embodiment of the present invention, the diameter of the ultrasonic probe 15 changes at greater than two defined intervals. In an embodiment of the present invention, the transitions 82 of the ultrasonic probe 15 are tapered to gradually change the diameter from the proximal end 31 to the distal end 24 along the longitudinal axis of the ultrasonic probe 15. In another embodiment of the present invention, the transitions 82 of the ultrasonic probe 15 are stepwise to change the diameter from the proximal end 31 to the distal end 24 along the longitudinal axis of the ultrasonic probe 15. Those skilled in the art will recognize there can be any number of defined intervals and transitions, and the transitions can be of any shape known in the art and be within the spirit and scope of the present invention.

In an embodiment of the present invention, the gradual change of the diameter from the proximal end 31 to the distal end 24 occurs over the at least one transition 82, with each transition 82 having an approximately equal length. In another embodiment of the present invention, the gradual change of the diameter from the proximal end 31 to the distal end 24 occurs over a plurality of transitions 82 with each transition 82 having a varying length. The transition 82 refers to a section where the diameter varies from a first diameter to a second diameter.

In a preferred embodiment of the present invention, the ultrasonic probe 15 has a small diameter. In a preferred embodiment of the present invention, the cross section of the ultrasonic probe 15 is approximately circular. In another embodiment, the cross section of at least a portion of the ultrasonic probe 15 is non-circular. The ultrasonic probe 15 comprising a wire having a non-circular cross section at the distal end can navigate through the vasculature. The ultrasonic probe 15 comprising a flat wire is steerable in the vasculature. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.1025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

The probe tip 9 can be any shape including, but not limited to, rounded, bent, a ball or larger shapes. In a preferred embodiment of the present invention, the probe tip 9 is atraumatic and smooth to prevent damage to the peripheral arteries. In one embodiment of the present invention, the ultrasonic energy source 99 is a physical part of the ultrasonic medical device 11. In another embodiment of the present invention, the ultrasonic energy source 99 is not an integral part of the ultrasonic medical device 11. The ultrasonic probe 15 is used to ablate the occlusional deposit 55 and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

The ultrasonic probe 15 is designed, constructed and comprised of a material to not dampen the transverse ultrasonic vibration, and thereby supports a transverse vibration when flexed. In a preferred embodiment of the present invention, the ultrasonic probe 15 comprises titanium or a titanium alloy. Titanium is a strong, flexible, low density, low radiopacity and easily fabricated metal that is used as a structural material. Titanium and its alloys have excellent corrosion resistance in many environments and have good elevated temperature properties. In a preferred embodiment of the present invention, the ultrasonic probe 15 comprises titanium alloy Ti-6Al-4V. The elements comprising Ti-6Al-4V and the representative elemental weight percentages of Ti-6Al-4V are titanium (about 90%), aluminum (about 6%), vanadium (about 4%), iron (maximum about 0.25%) and oxygen (maximum about 0.2%). In another embodiment of the present invention, the ultrasonic probe 15 comprises stainless steel. In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel.

In another embodiment of the present invention, the ultrasonic probe 15 comprises a super-elastic alloy. Even when bent or stretched, the super-elastic alloy returns to its original shape when the stress is removed. The ultrasonic probe 15 may comprise super-elastic alloys known in the art including, but not limited to, nickel-titanium super-elastic alloys and Nitinol. Nitinol is a family of intermetallic materials, which contain a nearly equal mixture of nickel and titanium. Other elements can be added to adjust or tune the material properties. Nitinol is less stiff than titanium and is maneuverable in the vasculature. Nitonol has shape memory and super-elastic characteristics. The shape memory effect describes the process of restoring the original shape of a plastically deformed sample by heating it. This is a result of a crystalline phase change known as thermoelastic martensitic transformation. Below the transformation temperature, Nitinol is martensitic. Nitinol's excellent corrosion resistance, biocompatibility, and unique mechanical properties make it well suited for medical devices. Those skilled in the art will recognize that the ultrasonic probe can be comprised of many other materials known in the art and be within the spirit and scope of the present invention.

The physical properties (i.e., length, cross sectional shape, dimensions, etc.) and material properties (i.e., yield strength, modulus, etc.) of the ultrasonic probe 15 are selected for operation of the ultrasonic probe 15 in the transverse mode. The length of the ultrasonic probe 15 of the present invention is chosen to be resonant in a transverse mode. In an embodiment of the present invention, the ultrasonic probe 15 is between about 30 centimeters and about 300 centimeters in length. Those skilled in the art will recognize an ultrasonic probe can have a length shorter than about 30 centimeters, a length longer than about 300 centimeters and a length between about 30 centimeters and about 300 centimeters and be within the spirit and scope of the present invention.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer is capable of an acoustic impedance transformation of electrical energy provided by the ultrasonic energy source 99 to mechanical energy. The transducer sets the operating frequency of the ultrasonic medical device 11. The transducer is capable of engaging the ultrasonic probe 15 at the proximal end 31 with sufficient restraint to form an acoustical mass that can propagate the ultrasonic energy provided by the ultrasonic energy source 99.

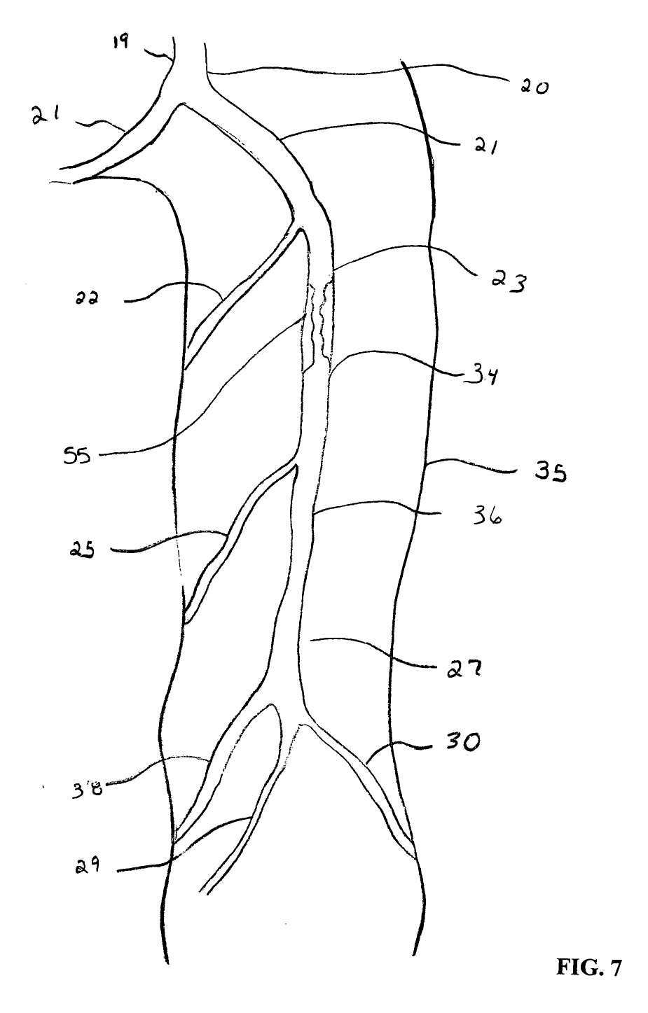

FIG. 7 shows a partial anatomy of the leg 35 having the occlusional deposit 55 in the external iliac artery 23. As discussed above, peripheral artery disease can occur in peripheral arteries located throughout the body. The present invention can be used to treat peripheral artery disease anywhere in the body including, but not limited to, the extremity limbs (i.e., the legs, feet, arms and hands), heart and brain. Those skilled in the art will recognize the ultrasonic probe 15 can be used to ablate occlusional deposits causing peripheral artery disease in the vasculature in other parts of the body and be within the spirit and scope of the present invention. Subsequent discussion of peripheral artery disease will focus on peripheral artery disease in the leg 35 of the patient, and more specifically in the external iliac artery 23, but the discussion is applicable to treatment of peripheral artery disease throughout the body.

As shown in FIG. 7, the abdominal aorta 20 bifurcates at an iliac bifurcation 19 into common iliac arteries 21. The common iliac arteries 21 separate into the internal iliac arteries 22 and continue on as the external iliac arteries 23. The internal iliac arteries 22 supply the pelvis with blood and oxygen. After passing under the inguinal ligament, the external iliac arteries 23 become the common femoral arteries 34. The common femoral artery 34 branches into the profunda femoral artery 25 as the superficial femoral artery 36 continues and passes into the popliteal fossa where the superficial femoral artery 36 is renamed the popliteal artery 27. After exiting the popliteal fossa, the popliteal artery 27 trifurcates into the anterior tibialis artery 38, the posterior tibialis artery 30 and the peroneal artery 29.

As discussed above, peripheral artery disease is a form of atherosclerosis in which the occlusional deposits 55 build up along the artery walls resulting in damage to the vessel wall by atheroma. Atheroma is a fatty deposit in the intima of the artery resulting from atherosclerosis. Peripheral artery disease is specific to partially or totally occlusive diseases of the peripheral arteries. When the buildup of the occlusional deposits 55 commences, there is an increase in vessel resistance that leads to a reduction in distal perfusion pressure and blood flow. As the occlusional deposits 55 build up, the effective radius of the afflicted peripheral artery is decreased. Despite atherosclerosis being a diffuse process that affects all of the arteries to some degree, some peripheral arteries in the particular limb may undergo greater stenosis than others.

The decrease in the effective radius of the peripheral artery increases the resistance to the fourth power of the change in radius. Resistance in the peripheral artery segment is directly proportional to the length of the peripheral artery segment and the viscosity of the blood and inversely proportional to the radius to the fourth power. In other words, a fifty percent reduction in radius causes the blood flow through the peripheral artery segment to decrease by a factor of sixteen. Equation 1 shows the relationship between the resistance to blood flow (R), the length of the peripheral artery segment (L), the viscosity of the blood (η) and the radius of the peripheral artery segment (r).

$$R ∝ \frac{η L}{{r}^{4}} \qquad Equation 1$$

If the above expression for resistance shown in Equation 1 is combined with the relationship between flow (F), pressure (ΔP) and resistance (R) shown in Equation 2,
F=ΔP/R Equation 2
then the result is Poiseuille's equation shown below as Equation 3.

$$F ∝ \frac{Δ P \cdot {r}^{4}}{η \cdot L} \qquad Equation 3$$

From a hydrodynamic standpoint, the decrease in flow by a factor of sixteen assumes laminar flow conditions, a constant pressure gradient and the peripheral artery segment is not one of multiple in series segments. However, since major arteries of limb circulation are both in series and in parallel, a stenotic lesion would have to have its radius decreased by more than sixty percent to result in a significant hydrodynamic effect such as a critical stenosis. Also, the presence of turbulence in the peripheral artery segment enhances the longitudinal pressure drop across the length of the occlusional deposit for any reduction in radius of the peripheral artery segment.

The peripheral artery disease leads to ischemia of the limb, where the limb does not receive an adequate oxygen supply. During exercise of the particular limb, the increased resistance to blood flow leads to decreased flow capacity through the peripheral artery, known as decreased active hyperemia. This condition results in ischemic pain known as intermittent claudication, a pain caused by tissue hypoxia resulting from the high oxygen demand that is not met by an adequate increase in the delivery of oxygen through increased blood flow. This condition results in a reduction in the oxygen supply/demand ratio. Metabolites formed under anaerobic conditions in the muscle stimulate pain receptors in the muscle.

In a preferred embodiment of the present invention, the access to the occlusional deposit 55 is from the side opposite of the occlusional deposit 55, an approach known as the contralateral approach. For example, in the embodiments of the present invention shown in FIG. 1 and FIG. 2, access is gained through the femoral artery 34 in the leg 56 and moved to the occlusional deposit 55 in the opposite leg 35. In another embodiment of the present invention shown in FIG. 3, access to the occlusional deposit 55 is from the same side as the occlusional deposit 55, an approach known as the ipsilateral approach.

In another embodiment of the present invention, access is gained through the iliac artery 21 of the leg 56. In another embodiment of the present invention, access is gained through the popliteal artery 27 of the leg 56. Those skilled in the art will recognize access can be gained in various peripheral arteries and be within the spirit and scope of the present invention.

In one embodiment of the present invention for the contralateral approach, a femoral puncture is made after locating the femoral artery 34 in the leg 56 opposite of the occlusional deposit 55. The puncture creates an insertion point in the femoral artery 34 in the leg 56. A guidewire is deployed in the femoral artery 34 and passed up and over the iliac bifurcation and down toward the occlusional deposit 55. In an embodiment of the present invention, the ultrasonic probe 15 is used in place of the guidewire. An introducer is inserted over the standard guidewire through the femoral puncture and a guide catheter is advanced within the introducer and moved proximal to the occlusional deposit 55. In an embodiment of the present invention, contrast is injected into the femoral artery 34 to help locate the occlusional deposit 55. The ultrasonic probe 15 of the present invention is inserted into the femoral puncture and placed across the occlusional deposit 55. In one embodiment of the present invention, the guide catheter is advanced across the occlusional deposit 55 and the guide catheter is pulled back to expose the ultrasonic probe 15. In another embodiment of the present invention, the ultrasonic probe 15 of the present invention is advanced across the occlusional deposit 55.

A device including, but not limited to, a vascular introducer can be used as the introducer to gain access to the peripheral artery. A vascular introducer for use with an ultrasonic probe is described in Assignee's co-pending patent application U.S. Ser. No. 10/080,787, and the entirety of this application is hereby incorporated herein by reference. In another embodiment of the present invention a sheath is used to gain access to the peripheral artery. In another embodiment of the present invention, a catheter is used to gain access to the peripheral artery.

In another embodiment of the present invention, the ultrasonic probe 15 of the present invention is advanced to the occlusional deposit 55 without using an introducer, sheath or catheter. Those skilled in the art will recognize the ultrasonic probe can be advanced adjacent the occlusional deposit in many ways known in the art and be within the spirit and scope of the present invention.

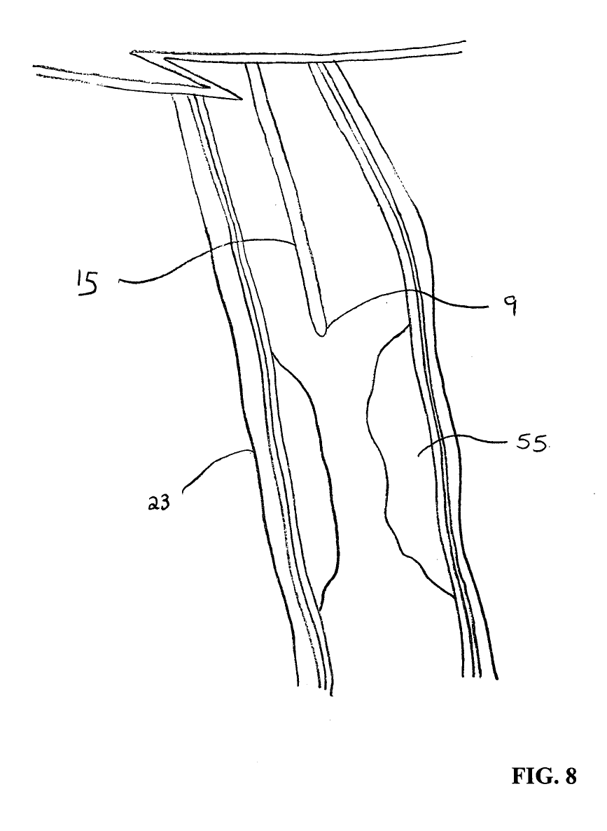

FIG. 8 shows an enlarged view of a portion of the ultrasonic probe 15 of the present invention inserted into the external iliac artery 23 of the leg 35 being moved toward the occlusional deposit 55. The ultrasonic probe 15 has a stiffness that gives the ultrasonic probe 15 a flexibility allowing the ultrasonic probe 15 to be deflected, flexed and bent through the tortuous paths of the peripheral arteries. The ultrasonic probe 15 can be bent, flexed and deflected to reach the occlusional deposit 55 in the peripheral arteries that would otherwise be difficult to reach. The stiffness of the ultrasonic probe 15 allows for the contralateral approach of the ultrasonic probe 15 without deforming the ultrasonic probe 15, damaging the vasculature the ultrasonic probe 15 is moving through, or dampening vibrations of the ultrasonic probe 15.

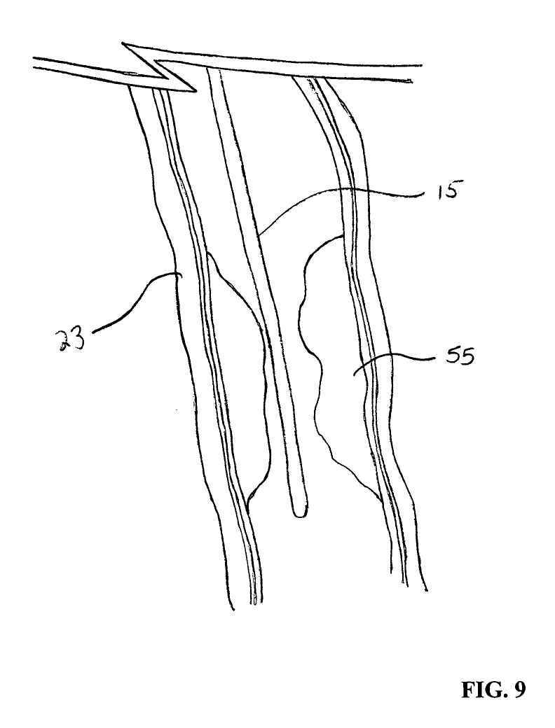

FIG. 9 shows an enlarged view of a portion of the longitudinal axis of the ultrasonic probe 15 in communication with the occlusional deposit 55 in the external iliac artery 23 of the leg 35. The ultrasonic probe 15 is moved within the external artery and placed in communication with the occlusional deposit 55. In an embodiment of the present invention, the ultrasonic probe 15 is used to create a channel through the occlusional deposit 55. In another embodiment of the present invention, a pharmacological agent is injected into the peripheral artery to soften or break down the occlusional deposit 55. An apparatus and a method for an ultrasonic probe used with a pharmacological agent is described in Assignee's U.S. Pat. No. 6,733,451, and the entirety of this patent is hereby incorporated herein by reference.

In an embodiment of the present invention, the probe tip 9 comprises a material of high radiopacity or a radiopaque marker. In an embodiment of the present invention where a guide catheter is used, a distal end of the guide catheter comprises a material of high radiopacity or a radiopaque marker. Under fluoroscopy, a treatment zone of the ultrasonic energy can be seen by the radiopaque tip 9 of the ultrasonic probe 15 and the radiopaque distal end of the guide catheter. A material of high radiopacity does not allow the passage of a substantial amount of x-rays or other radiation and therefore allows a higher degree of visibility in an imaging procedure. An ultrasonic medical device comprising a material of high radiopacity is described in Assignee's U.S. Pat. No. 6,730,048 and an ultrasonic medical device comprising a radiopaque marker is described in Assignee's co-pending patent application U.S. Ser. No. 10/207,468 (published patent application No. 2004/0019266), and the entirety of this patent and patent application are hereby incorporated herein by reference.

After the ultrasonic probe 15 is placed in communication with the occlusional deposit 55, the ultrasonic energy source 99 is activated to provide a low power electric signal of between about 2 watts to about 15 watts to the transducer that is located within the handle 88. The transducer converts electrical energy provided by the ultrasonic energy source 99 to mechanical energy. The operating frequency of the ultrasonic medical device 11 is set by the transducer and the ultrasonic energy source 99 finds the resonant frequency of the transducer through a Phase Lock Loop. By an appropriately oriented and driven cylindrical array of piezoelectric crystals of the transducer, the horn creates a longitudinal wave along at least a portion of the longitudinal axis of the ultrasonic probe 15. The longitudinal wave is converted to a transverse wave along at least a portion of the longitudinal axis of the ultrasonic probe 15 through a nonlinear dynamic buckling of the ultrasonic probe 15.

As the transverse wave is transmitted along the longitudinal axis of the ultrasonic probe 15, a transverse ultrasonic vibration is created along the longitudinal axis of the ultrasonic probe 15. The ultrasonic probe 15 is vibrated in a transverse mode of vibration. The transverse mode of vibration of the ultrasonic probe 15 differs from an axial (or longitudinal) mode of vibration disclosed in the prior art. The transverse ultrasonic vibrations along the longitudinal axis of the ultrasonic probe 15 create a plurality of transverse nodes and a plurality of transverse anti-nodes along a portion of the longitudinal axis of the ultrasonic probe 15.

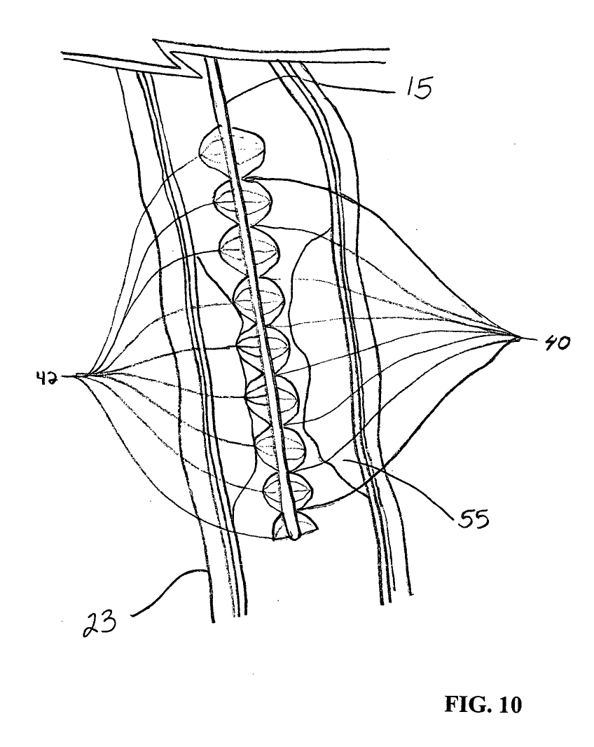

FIG. 10 shows the ultrasonic probe 15 of the present invention having a plurality of transverse nodes 40 and a plurality of transverse anti-nodes 42 along a portion of the longitudinal axis of the ultrasonic probe 15 and in communication with the occlusional deposit 55. The transverse nodes 40 are areas of minimum energy and minimum vibration that occur at repeating intervals along the portion of the longitudinal axis of the ultrasonic probe 15. The transverse anti-nodes 42, or areas of maximum energy and maximum vibration, occur at repeating intervals along the portion of the longitudinal axis of the ultrasonic probe 15. The number of transverse nodes 40 and transverse anti-nodes 42, and the spacing of the transverse nodes 40 and transverse anti-nodes 42 of the ultrasonic probe 15 depend on the frequency of energy produced by the ultrasonic energy source 99. The separation of the transverse nodes 40 and transverse anti-nodes 42 is a function of the frequency, and can be affected by tuning the ultrasonic probe 15. In a properly tuned ultrasonic probe 15, the transverse anti-nodes 42 will be found at a position one-half of the distance between the transverse nodes 40 located adjacent to each side of the transverse anti-nodes 42.

The transverse wave is transmitted along the longitudinal axis of the ultrasonic probe 15 and the interaction of the surface of the ultrasonic probe 15 with the medium surrounding the ultrasonic probe 15 creates an acoustic wave in the surrounding medium. As the transverse wave is transmitted along the longitudinal axis of the ultrasonic probe 15, the ultrasonic probe 15 vibrates transversely. The transverse motion of the ultrasonic probe 15 produces cavitation in the medium surrounding the ultrasonic probe 15 to ablate the occlusional deposit 55. Cavitation is a process in which small voids are formed in a surrounding medium through the rapid motion of the ultrasonic probe 15 and the voids are subsequently forced to compress. The compression of the voids creates a wave of acoustic energy which acts to dissolve the matrix binding the occlusional deposit 55, while having no damaging effects on healthy tissue.

The occlusional deposit 55 in the peripheral artery is resolved into a particulate having a size on the order of red blood cells (approximately 5 microns in diameter). The size of the particulate is such that the particulate is easily discharged from the body through conventional methods or simply dissolves into the blood stream. A conventional method of discharging the particulate from the body includes transferring the particulate through the blood stream to the kidney where the particulate is excreted as bodily waste.

The transverse ultrasonic vibration of the ultrasonic probe 15 results in a portion of the longitudinal axis of the ultrasonic probe 15 vibrated in a direction not parallel to the longitudinal axis of the ultrasonic probe 15. The transverse vibration results in movement of the longitudinal axis of the ultrasonic probe 15 in a direction approximately perpendicular to the longitudinal axis of the ultrasonic probe 15. Transversely vibrating ultrasonic probes for biological material ablation are described in the Assignee's U.S. Pat. No. 6,551,337; U.S. Pat. No. 6,652,547; U.S. Pat. No. 6,660,013; and U.S. Pat. No. 6,695,781, which further describe the design parameters for such an ultrasonic probe and its use in ultrasonic devices for ablation, and the entirety of these patents are hereby incorporated herein by reference.

As a consequence of the transverse ultrasonic vibration of the ultrasonic probe 15, the occlusional deposit destroying effects of the ultrasonic medical device 11 are not limited to those regions of the ultrasonic probe 15 that may come into contact with the occlusional deposit 55. Rather, as a section of the longitudinal axis of the ultrasonic probe 15 is positioned in proximity to the occlusional deposit 55, the occlusional deposit 55 is removed in all areas adjacent to the plurality of energetic transverse anti-nodes 42 that are produced along the portion of the length of the longitudinal axis of the ultrasonic probe 15, typically in a region having a radius of up to about 6 mm around the ultrasonic probe 15.

A novel feature of the present invention is the ability to utilize ultrasonic probes 15 of extremely small diameter compared to prior art probes, without loss of efficiency, because the occlusional deposit fragmentation process is not dependent on the area of the probe tip 9. Highly flexible ultrasonic probes 15 can therefore be designed for facile insertion into occluded areas or extremely narrow interstices that contain the occlusional deposit 55. Another advantage provided by the present invention is the ability to rapidly remove the occlusional deposit 55 from large areas within cylindrical or tubular surfaces.

The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of occlusional deposit destroying transverse anti-nodes 42 along the longitudinal axis of the ultrasonic probe 15. The low frequency requirement of the present invention is a further advantage in that the low frequency requirement leads to less damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to the occlusional deposit 55 selectively, because the ultrasonic probe 15 conducts energy across a frequency range from about 10 kHz through about 100 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 10 kHz to about 100 kHz. In a preferred embodiment of the present invention, the frequency of ultrasonic energy is from about 20 kHz to about 40 kHz.

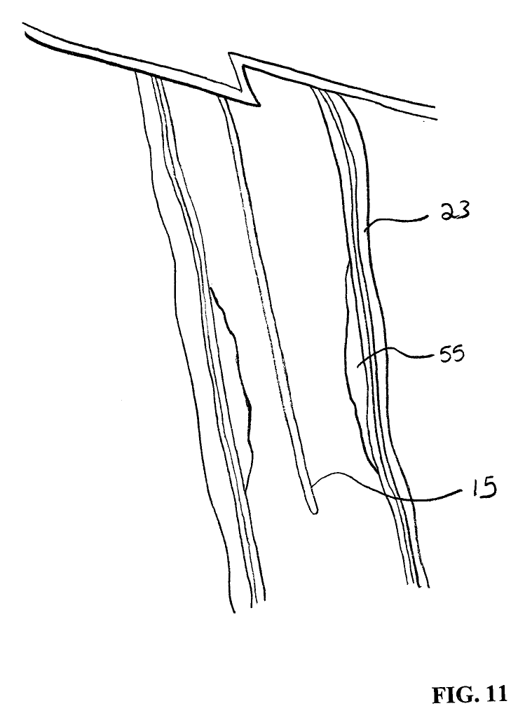

FIG. 11 shows an enlarged view of the ultrasonic probe 15 of the present invention in communication with a partially ablated occlusional deposit 55 in the external iliac artery 23. As described above, the transverse motion of the ultrasonic probe 15 produces cavitation in the medium surrounding the ultrasonic probe 15 to ablate the occlusional deposit 55. FIG. 11 is a view of the ablation process where the entire occlusional deposit 55 causing the peripheral artery disease is removed.

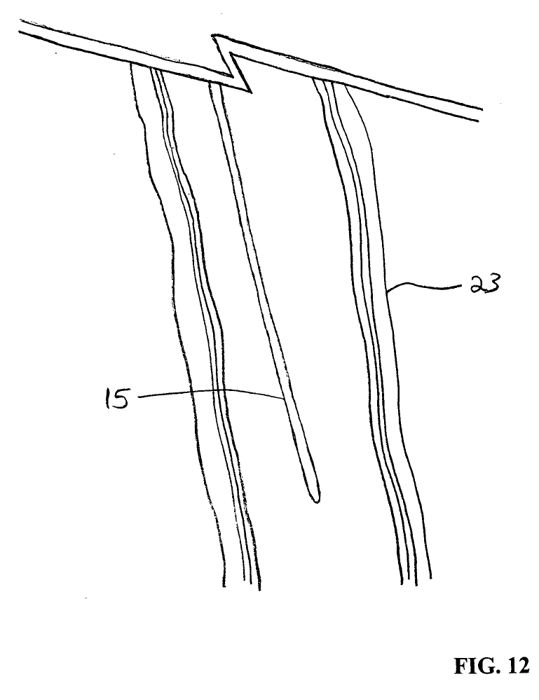

FIG. 12 shows an enlarged view of the external iliac artery 23 with the occlusional deposit 55 removed after treatment with the ultrasonic medical device 11 of the present invention. In an embodiment of the present invention, a residual portion of the occlusional deposit 55 remains in the vasculature after treatment with the ultrasonic medical device 11. In another embodiment of the present invention, the occlusional deposit 55 is removed and does not remain in the vasculature after treatment with the ultrasonic medical device 11. Those skilled in the art will recognize that varying amounts of residual occlusional deposit can remain in t the vasculature and still be within the spirit and scope of the present invention.

The present invention also provides a method of treating peripheral artery disease. For the contralateral approach, a medical professional gains access to the peripheral artery in the leg 35 through an insertion point or puncture in the peripheral artery in the opposite leg having the occlusional deposit 55. For the ipsilateral approach, the insertion point is in the peripheral artery on same leg having the occlusional deposit 55. The ultrasonic probe 15 of the present invention is inserted into the vasculature and moved up and across the iliac bifurcation and adjacent to the occlusional deposit 55. The ultrasonic probe 15 is placed in communication with the occlusional deposit 55 by moving, sweeping, twisting, bending or rotating the ultrasonic probe 15 along the occlusional deposit 55. The ultrasonic probe 15 is placed in communication with the occlusional deposit 55 and the ultrasonic energy source 99 engaged to the ultrasonic probe 15 is activated to generate a transverse ultrasonic vibration along at least a portion of the longitudinal axis of the ultrasonic probe 15. The transverse ultrasonic vibration creates a plurality of transverse nodes 40 and a plurality of transverse anti-nodes 42 along a portion of the longitudinal axis of the ultrasonic probe 15.

The present invention also provides a method of treating peripheral artery disease to decrease the vessel resistance resulting from the buildup of the occlusional deposit 55 along the inner walls of the vasculature. The present invention provides a method of resolving the occlusional deposit 55 to a particulate so the effective radius of the afflicted peripheral artery is returned to the radius without the buildup of the occlusional deposit. Access to a peripheral artery is gained and the ultrasonic probe 15 is inserted into an insertion point in the leg 56 opposite of the leg 35 having the occlusional deposit 55 (contralateral approach) or the insertion point is in the same leg having the occlusional deposit 55 (ipsilateral approach). The ultrasonic probe 15 is moved adjacent to the occlusional deposit 55 and placed in communication with the occlusional deposit 55. The ultrasonic energy source 99 engaged to the ultrasonic probe 15 produces an electric signal that drives a transducer of the ultrasonic medical device to produce a transverse ultrasonic vibration of the ultrasonic probe 15 that produces cavitation in a medium surrounding the ultrasonic probe 15 to ablate the occlusional deposit 55.

In an alternative embodiment of the present invention, the ultrasonic probe 15 is vibrated in a torsional mode. In the torsional mode of vibration, a portion of the longitudinal axis of the ultrasonic probe 15 comprises a radially asymmetric cross section and the length of the ultrasonic probe 15 is chosen to be resonant in the torsional mode. In the torsional mode of vibration, a transducer transmits ultrasonic energy received from the ultrasonic energy source 99 to the ultrasonic probe 15, causing the ultrasonic probe 15 to vibrate torsionally. The ultrasonic energy source 99 produces the electrical energy that is used to produce a torsional vibration along the longitudinal axis of the ultrasonic probe 15. The torsional vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of a plurality of torsional nodes and a section distal to each of the plurality of torsional nodes are vibrated out of phase, with the proximal section vibrated in a clockwise direction and the distal section vibrated in a counterclockwise direction, or vice versa. The torsional vibration results in an ultrasonic energy transfer to the biological material with minimal loss of ultrasonic energy that could limit the effectiveness of the ultrasonic medical device 11. The torsional vibration produces a rotation and a counterrotation along the longitudinal axis of the ultrasonic probe 15 that creates the plurality of torsional nodes and a plurality of torsional anti-nodes along a portion of the longitudinal axis of the ultrasonic probe 15 resulting in cavitation along the portion of the longitudinal axis of the ultrasonic probe 15 comprising the radially asymmetric cross section in a medium surrounding the ultrasonic probe 15 that ablates the biological material. An apparatus and method for an ultrasonic medical device operating in a torsional mode is described in Assignee's co-pending patent application U.S. Ser. No. 10/774,985, and the entirety of this application is hereby incorporated herein by reference.

In another embodiment of the present invention, the ultrasonic probe 15 is vibrated in a torsional mode and a transverse mode. A transducer transmits ultrasonic energy from the ultrasonic energy source 99 to the ultrasonic probe 15, creating a torsional vibration of the ultrasonic probe 15. The torsional vibration induces a transverse vibration along an active area of the ultrasonic probe 15, creating a plurality of nodes and a plurality of anti-nodes along the active area that result in cavitation in a medium surrounding the ultrasonic probe 15. The active area of the ultrasonic probe 15 undergoes both the torsional vibration and the transverse vibration.

Depending upon physical properties (i.e., length, diameter, etc.) and material properties (i.e., yield strength, modulus, etc.) of the ultrasonic probe 15, the transverse vibration is excited by the torsional vibration. Coupling of the torsional mode of vibration and the transverse mode of vibration is possible because of common shear components for the elastic forces. The transverse vibration is induced when the frequency of the transducer is close to a transverse resonant frequency of the ultrasonic probe 15. The combination of the torsional mode of vibration and the transverse mode of vibration is possible because for each torsional mode of vibration, there are many close transverse modes of vibration. By applying tension on the ultrasonic probe 15, for example by bending the ultrasonic probe 15, the transverse vibration is tuned into coincidence with the torsional vibration. The bending causes a shift in frequency due to changes in tension. In the torsional mode of vibration and the transverse mode of vibration, the active area of the ultrasonic probe 15 is vibrated in a direction not parallel to the longitudinal axis of the ultrasonic probe 15 while equally spaced points along the longitudinal axis of the ultrasonic probe 15 vibrate back and forth in a short arc about the longitudinal axis of the ultrasonic probe 15. An apparatus and method for an ultrasonic medical device operating in a transverse mode and a torsional mode is described in Assignee's co-pending patent application U.S. Ser. No. 10/774,898, and the entirety of this application is hereby incorporated herein by reference.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An ultrasonic medical device for treating peripheral artery disease comprising:
an ultrasonic probe having a proximal end, a distal end and a longitudinal axis between the proximal end and the distal end;
a transducer creating a transverse ultrasonic vibration along at least a portion of the longitudinal axis of the ultrasonic probe;
a coupling engaging the proximal end of the ultrasonic probe to a distal end of the transducer; and
an ultrasonic energy source engaged to the transducer that produces an ultrasonic energy,
wherein the transverse ultrasonic vibration produces a plurality of transverse anti-nodes along at least a portion of the longitudinal axis of the ultrasonic probe to ablate an occlusional deposit of peripheral artery disease.

2. The ultrasonic medical device of claim 1 wherein the ultrasonic probe has a flexibility allowing the ultrasonic probe to be deflected and articulated.

3. The ultrasonic medical device of claim 1 wherein the ultrasonic energy source delivers ultrasonic energy in a frequency range from about 10 kHz to about 100 kHz.

4. The ultrasonic medical device of claim 1 wherein the transverse ultrasonic vibration generates acoustic energy in a medium surrounding the ultrasonic probe.

5. The ultrasonic medical device of claim 1 wherein the transverse ultrasonic vibration along the longitudinal axis of the ultrasonic probe interacts with a medium surrounding the ultrasonic probe to create an acoustic wave in the medium.

6. The ultrasonic medical device of claim 1 wherein the ultrasonic energy source provides an electrical energy to the transducer.

7. The ultrasonic medical device of claim 1 wherein the ultrasonic energy source provides an electrical energy to the transducer at a resonant frequency of the transducer by finding the resonant frequency of the transducer.

8. The ultrasonic medical device of claim 1 wherein the ultrasonic probe supports the transverse ultrasonic vibration when flexed.

9. The ultrasonic medical device of claim 1 wherein the transverse ultrasonic vibration produces a plurality of transverse nodes along at least a portion of the longitudinal axis of the ultrasonic probe.

10. The ultrasonic medical device of claim 1 wherein the transverse ultrasonic vibration of the ultrasonic probe produces cavitation in a medium surrounding the ultrasonic probe to ablate the occlusional deposit to treat peripheral artery disease.

11. The ultrasonic medical device of claim 1 wherein the ultrasonic probe is disposable.

12. The ultrasonic medical device of claim 1 wherein the ultrasonic probe is for a single use on a single patient.

13. An ultrasonic medical device for ablating an occlusional deposit of peripheral artery disease comprising:
an ultrasonic probe having a proximal end, a distal end terminating in a probe tip and a longitudinal axis between the proximal end and the distal end;
a transducer that converts electrical energy into mechanical energy, creating a transverse ultrasonic vibration along the longitudinal axis of the ultrasonic probe;
and
a coupling engaging the proximal end of the ultrasonic probe and a distal end of the transducer,
wherein the transverse ultrasonic vibration generates a plurality of transverse anti-nodes along at least a portion of the longitudinal axis of the ultrasonic probe, creating cavitation in a medium surrounding the ultrasonic probe to ablate the occlusional deposit to treat peripheral artery disease.

14. The ultrasonic medical device of claim 13 wherein the transverse ultrasonic vibration generates acoustic energy in a medium surrounding the ultrasonic probe.

15. The ultrasonic medical device of claim 13 wherein the ultrasonic probe comprises a diameter that enables insertion into a vasculature.

16. The ultrasonic medical device of claim 13 wherein the ultrasonic probe comprises a diameter that allows the ultrasonic probe to be bent, flexed and deflected.

17. The ultrasonic medical device of claim 13 wherein a diameter of the ultrasonic probe is uniform diameter from the proximal end to the distal end.

18. The ultrasonic medical device of claim 13 wherein a diameter of the ultrasonic probe varies from the proximal end to the distal end.

19. The ultrasonic medical device of claim 13 wherein a cross section of the ultrasonic probe is approximately circular.

20. The ultrasonic medical device of claim 13 wherein a cross section of at least a portion of the ultrasonic probe is non-circular.

21. The ultrasonic medical device of claim 13 wherein the transverse ultrasonic vibration generates a plurality of transverse nodes along at least a portion of the longitudinal axis of the ultrasonic probe.

22. A method of treating peripheral artery disease comprising:
providing an ultrasonic medical device comprising an ultrasonic probe having a proximal end, a distal end terminating in a probe tip and a longitudinal axis between the proximal end and the distal end;
inserting the ultrasonic probe into a vasculature;
moving the ultrasonic probe adjacent to an occlusional deposit;
placing the ultrasonic probe in communication with the occlusional deposit; and
activating an ultrasonic energy source engaged to the ultrasonic probe to generate a transverse ultrasonic vibration along at least a portion of the longitudinal axis of the ultrasonic probe,
wherein the transverse ultrasonic vibration creates a plurality of transverse anti-nodes along a portion of the longitudinal axis of the ultrasonic probe.

23. The method of claim 22 further comprising creating a channel through the occlusional deposit with the ultrasonic probe.

24. The method of claim 22 further comprising puncturing a femoral artery to gain access to the vasculature.

25. The method of claim 22 further comprising puncturing a femoral artery in a leg having the occlusional deposit for an ipsilateral approach.

26. The method of claim 22 further comprising puncturing a femoral artery in a leg opposite a leg having the occlusional deposit for a contralateral approach.

27. The method of claim 22 further comprising accessing the vasculature with an introducer.

28. The method of claim 22 further comprising accessing the vasculature with a sheath.

29. The method of claim 22 further comprising accessing the vasculature with a catheter.

30. The method of claim 22 further comprising transmitting a transverse wave from the transverse ultrasonic vibration along the longitudinal axis of the ultrasonic probe to create an acoustic wave in the medium surrounding the ultrasonic probe.

31. The method of claim 22 further comprising delivering ultrasonic energy in a frequency range of about 10 kHz to about 100 kHz by the ultrasonic energy source.

32. The method of claim 22 further comprising generating acoustic energy in a medium surrounding the ultrasonic probe through the transverse ultrasonic vibration of the ultrasonic probe.

33. The method of claim 22 further comprising moving the ultrasonic probe back and forth along the occlusional deposit.

34. The method of claim 22 further comprising rotating the ultrasonic probe along the occlusional deposit.

35. The method of claim 22 further comprising sweeping the ultrasonic probe along the occlusional deposit.

36. The method of claim 22 further comprising providing an electrical energy to a transducer at a resonant frequency of the transducer by the ultrasonic energy source determining the resonant frequency of the transducer.

37. The method of claim 22 further comprising creating a plurality of transverse nodes from the transverse ultrasonic vibration along a portion of the longitudinal axis of the ultrasonic probe.

38. A method of ablating an occlusional deposit to treat peripheral artery disease comprising:
providing an ultrasonic medical device comprising an ultrasonic probe having a proximal end, a distal end and a longitudinal axis between the proximal end and the distal end;
inserting the ultrasonic probe into a femoral artery;
moving the ultrasonic probe into a peripheral artery;
placing the ultrasonic probe in communication with an occlusional deposit in the peripheral artery; and
activating an ultrasonic energy source engaged to the ultrasonic probe to produce an electric signal that drives a transducer of the ultrasonic medical device to produce a transverse ultrasonic vibration of the ultrasonic probe,
wherein the transverse ultrasonic vibration produces cavitation in a medium surrounding the ultrasonic probe to ablate the occlusional deposit.

39. The method of claim 38 further comprising transmitting a transverse wave from the transverse ultrasonic vibration along the longitudinal axis of the ultrasonic probe to create an acoustic wave in the medium surrounding the ultrasonic probe.

40. The method of claim 38 further comprising producing a plurality of transverse nodes and a plurality of transverse anti-nodes along a portion of the longitudinal axis of the ultrasonic probe.

41. The method of claim 40 wherein the plurality of transverse nodes are points of a minimum transverse ultrasonic vibration.

42. The method of claim 40 wherein the plurality of transverse anti-nodes are points of a maximum transverse ultrasonic vibration.

43. The method of claim 38 further comprising sweeping the ultrasonic probe along the occlusional deposit.

44. The method of claim 38 further comprising moving the ultrasonic probe back and forth along the occlusional deposit.

45. The method of claim 38 further comprising sweeping the ultrasonic probe back and forth along the occlusional deposit.

46. The method of claim 38 further comprising puncturing a femoral artery to gain access to a vasculature.

47. The method of claim 38 further comprising puncturing a femoral artery in a leg having the occlusional deposit for an ipsilateral approach.

48. The method of claim 38 further comprising puncturing a femoral artery in a leg opposite a leg having the occlusional deposit for a contralateral approach.
